(19) United States
(12) Patent Application Publication
(10) Pub. No.: US 2023/0408509 A1
(43) Pub. Date: Dec. 21, 2023

(54) Automatic Diagnostic Tracing

(71) Applicant: LMX MedTech LLC, Bloomfield, MI (US)

(72) Inventors: Michael J. Pugia, Pagosa Springs, CO (US); Jason M. Kulick, South Bend, IN (US)

(21) Appl. No.: 18/249,078

(22) PCT Filed: Oct. 14, 2021
(86) PCT No.: PCT/US21/54998

§ 371 Date: Apr. 14, 2023

Related U.S. Application Data
(60) Provisional application No. 63/092,890, filed on Oct. 16, 2020.

(51) Int. Cl. G01N 33/543; G01N 27/447; G01N 27/327; G01N 27/416; G01N 33/569

(52) U.S. Cl. CPC G01N 33/5438; G01N 27/44791; G01N 27/44743; G01N 27/3276; G01N 27/4163; G01N 33/56911

(57) ABSTRACT

A method of using electrochemical reporters in a micro-filtration sensor which contains multiple analyte detection wells with electrodes and where the electrochemical reporters determine the identity and integrity of the product and sample. Identity and integrity are assessed for suitable use and linkage to additional data or preventing producing erroneous in-vitro diagnostic data. The micro-filtration sensor is additionally used for electrochemical bioassays of analytes in one or more of the analyte detection microwells. Analyte detection microwells may contain an additional affinity agent for capture of affinity agents for analyte detection and electrochemical reporters for identity and integrity detection.

### CROSS REFERENCE TO RELATED APPLICATIONS

This application is the United States national phase of International Application No. PCT/US21/54998 filed Oct. 14, 2021, and claims priority to U.S. Provisional Patent Application No. 63/092,890, filed Oct. 16, 2020, the disclosures of which are hereby incorporated by reference in their entireties.

### BACKGROUND OF THE INVENTION

### Field of the Invention

The present disclosure relates to a method of using electronic reporters of product and sample identity on a reagent, such as a micro sensor array. Such method is used to verify the integrity of produced values, which are capable for electronic calibration and the integrity of results, such as bioassay producing in-vitro diagnostic data, which can be linked to archival sample and to additional data, such as the identity of the individual or organism sampled, sampling time and other data related to the results.

### Description of Related Art

Diagnostic laboratory systems are currently unable to decrease the time between the collection of an in-vitro sample and the time to return the diagnostic information for remedial action from better than 24 to 72 hours. This is due to the size of the equipment, the skill level needed to operate and the methods of collecting the biological sample. Point of care (POC) diagnostics systems can collect in-vitro diagnostic data directly from an individual's sample by immediately processing complex samples and determining the amounts of rare molecules. These systems decrease the time from collection to remedial action to less than 30 minutes (Pugia Anal Chem 2021).

Current POC systems require better verification of results and better data connections between the reagents and the analyzer to automatically identify product integrity. Several improvements have been made in the prior art. In Corey et al., U.S. Pat. No. 6,316,264, the use of an optical reporter on test strips for bioassay was demonstrated to detect validity of the diagnostic result by indicating when a strip was not properly placed in the system. In Alberella et al., U.S. Pat. No. 6,673,630, the use of optical reporters on test strips was demonstrated to provide a quality check of the system and reagent performance status as needed. In Zimmerle et al., U.S. Pat. No. 9,145,576, the use of an optical reporter on test strips was demonstrated to show moisture damage of the strip. In Howard III et al., U.S. Pat. No. 5,945,341, the use of optical reporters is often placed in a pre-defined location on a reagent disposable, such as a test strip or cartridge, to identify the reagent product automatically from a menu of potential reagent products.

Once the product identity has been verified, as it is also commonly known to those who practice in the art, a calibration code can be reported to an analyzer, such as through a bar code or RFID signal to provide additional calibration settings for that manufacturing lot. This code is often placed on the reagent as in a disposable form, such as a test strip, sensor, package, cartridge, or chip. These methods can automatically assign calibration settings to a system by providing data specific to the reagent lot from the time of manufacturing, which are traceable to standardization and allow calculation of analyte results. This is especially needed and used for quantitative bioassays, which require lot to lot adjustments.

Current POC systems need better methods to automatically identify and report integrity of samples capable of producing results. Several improvements have been made in the prior art. In Pugia, U.S. Pat. No. 5,374,561, the use of creatinine reagent on test strips for bioassay was demonstrated to detect validity of the urine sample as being sufficiently concentrated for producing a reagent result. In Kuo et al., U.S. Pat. No. 6,183,972, the use of optical reporters on immunochromatography test strips were demonstrated to provide a quality check of samples for correcting of a hook effect. In Pugia et al, Clin. Chem Lab Med. 2004, the use of optical reporters on immunochromatography test strips was demonstrated to provide a quality check of samples to a flow through device. However, these methods are not general to all sample types or issues, and do not correct for all interferents in all samples. This results in significant between-sample variability and all samples are not able to be automatically verified as being capable of producing accurate results.

A factor not addressed in the prior art is the ability to automatically connect the samples collected to: 1) reagent results; 2) an archived sample for repeat analysis; and 3) the additional data. This not only requires high density reading and writing of information but also requires a means to test the samples multiple times after collection, as well as automatic verification of the product and sample integrity and identity to produce results that are suitable for linking to additional data. Additional data can be any information, data, or record for the sample tested such as the identity of the sample, sampling time, origin of the sample such a patient, donor, organism, or environment and other data connected to the sample. Pugia Anal Chem 2021 demonstrates processes for collection of an initial sample that can be archived and allow repeat analysis using several types of immune and molecular assays. However, this process did not address product and sample integrity or suitability of reagent of the sample, or allowing automation of a system verification and calibration or for indicating suitable linking to additional data.

Improvements are needed for the method to upload bio-analysis data into the electronic medical record with a time and a patient record time stamp. This currently is performed routinely in clinical laboratories using diagnostics systems to upload data into the electronic medical record with a time and a patient record time stamp. Yet, this current method has noted the inability to trace or utilize in-vitro results over a time period, which is needed to measure the real time flux of biological systems. The inability of the current POC systems to verify product and sample integrity prohibits data connections between produced results and linking additional data.

Tracing the identity of organism producing samples over a given time period allows real time flux of the biological systems to be measured. Tracing one or more organisms or patients that are analyzed over time allows predictive models of biological systems for predicting complex outcomes. These outcomes can be used, for example, for the wellness of the host organism, managing the health of biomes of multiple organisms, improving the cost effectiveness of disease management, and protecting resources or containment of pathogenic organisms.

Diagnostic systems are currently unable to trace one or more organisms or access the time stamp for results from any given sample from an organism. This restricts the ability to improve the prediction of complex outcomes using algorithms developed by artificial intelligence and modeling. For example, the clinical outcome of a subject with a disease cannot be evaluated or improved to stall progression of disease over the life of the subject.

Currently, the clinical studies for evaluating disease progression of groups of subjects are lengthy and costly as many disease incidences or affect rates (e.g. death rate) are <5%/year, and it is difficult to predict the collection of samples and patients needed in advance. Assessments requiring lengthy sample collections of many subjects are often prohibitive and lengthy unless retrospective archival samples are used. Diagnostic systems able to identify the organisms providing the samples over time would significantly improve the predictions.

The protection and wellness of organisms and biomes often require diagnostic system data, which is timely and cost-effective, and provides answers for biological systems at the point where immediate actions can be taken. For example, an infectious disease spreading throughout a biome of multiple organisms often requires immediate isolation of one or more individual organisms to prevent spreading pathogens or destroying the wellness by causing imbalances of the biological systems (dysbiosis). The ability to read electronic records immediately and to trace or utilize in-vitro results over a time period is not currently possible with the current diagnostic systems.

IBRI's PCT/US2020/055931 (the “IBRI PCT”), which is incorporated by reference in its entirety, has recently demonstrated a device format that could perform multiplexed analysis of biomolecules directly on complex samples using multiple analyte detection microwells for electrochemical detection of target analytes. The analyte detection microwell includes a size exclusion filter with one or more pores, electrochemical detector, and affinity agents for a target analyte for capture and detection, which operates under a low hydrodynamic force. The affinity agent for detection is attached to a reagent capable of generating an electrochemical label. The affinity agent for capture is attached to a reagent capable of binding a surface in the microwell. The electrochemical label is detected by a working electrode and a counter/reference electrode placed in the microwell to measure labels formed by the affinity agent for detection. The format enables processing of biomolecule capture and immunoassay detection in a convenient format without needing user intervention to remove the filtration membrane to produce results.

The IBRI PCT design allows precise containment of small sample volumes in analyte detection microwells without loss of detection liquid, exposure to the environment, or the need for extraction and delivery into an analyzer. A microfluidic capillary stop placed underneath multiple microwells is used to hold the liquid in the microwells for capture and detection of the analyte to be measured. The device operates after manual dilution and mixing of the sample with liquids and affinity reagents, such as antibodies needed for immunoassays. The liquid reagents allow the diluted sample and reagents to be moved for capture and detection in the analyte detection microwell. Elimination of the manual dilution and mixing of the sample with liquids and affinity reagents is needed to allow the device to avoid any user intervention. Avoiding any user intervention is required for the design to be used in home testing and point-of-care testing settings. Avoiding the need for additional liquids, valves, and liquid dispensers is highly desired for miniaturization and ease of use.

The IBRI PCT device can be used with reagents for affinity assays such as electrochemical immunoassays (EC-IA), optical immunoassays (OP-IA), and mass spectrometric immunoassays (MS-IA) in the detection of cells and biomolecules trapped on the filtration membrane. In one example, polyclonal affinity reagents were used as a sandwich assay pair by placing an affinity label (biotin) on some of the polyclonal antibodies and placing a detection method (ALP) on the remaining polyclonal antibody. This format allows the biomolecules to be immediately captured on the filtration membrane using neutravidin attached to capture microparticles trapped on the membrane surface. For multiplexed analysis, the filtration membrane is divided into multiple microwells with a filtration membrane bottom. Descriptions of the affinity assay utilized may be found in Pugia, M. J. et al., “Multiplexed SIERRA Assay for the Culture-Free Detection of Gram-Negative and Gram-Positive Bacteria and Antimicrobial Resistance Genes” Anal Chem, 2021.

A solution is needed to allow identity and integrity of the product and sample to be verified that is easily integrated onto small reagent disposals of the IBRI PCT, and allow linking additional data to the analyte data collected from immediate testing or archival sample testing.

### SUMMARY OF THE INVENTION

An object of an embodiment of the present disclosure is to provide one or more electrochemical reporters into a micro-filtration sensor to determine if the identity and integrity of the product and sample are suitable for use and linked to additional data. The sensor array is, additionally, used for electrochemical detection of analytes and includes one or more analyte detection microwells, size exclusion filters, electrochemical detectors, and affinity agents for capture and detection of a target analyte. The affinity agent for analyte detection is attached to a reagent capable of generating an electrochemical signal. Additional affinity agents are placed in the sensor array to capture the affinity agents and electrochemical reporters.

In non-limiting embodiments or examples, electrochemical reporters in one or more analyte detection microwell with or without captured and/or detected analytes, and the microwell can be removed and stored for additional analysis. The electrochemical reporter is used to report identity and integrity of the results, can also be used to identify the archival analyte detection microwell with the identity of the sample and the sampling time.

In non-limiting embodiments or examples, the electrochemical reporters are attached to a reagent capable of being affinity agents for capture or attached to a reagent capable of binding a surface in the analyte detection microwell. The capture or attached electrochemical label is detected by a working electrode and a reference electrode placed in the microwell to measure the electrochemical reporter. The electrochemical reporter of product identity disclosed herein includes producing electrochemical signals for calibration of a sensor. In some non-limiting embodiments or examples, the electrochemical reporter of product identity is placed in a specific position on an array of one or more analyte detection microwells. The microwell position and electrochemical labels detected are used as an indicator of product identity. In other non-limiting embodiments or examples, the amount of electrochemical reporter is varied as an indicator of product identity by detection of the amount of electrochemical reporter.

In non-limiting embodiments or examples, the affinity agents for capture of target analyte are not included in the sensor with the electrochemical reporter of the product identity and integrity. The electrochemical reporter is captured after the addition of the liquid to the analyte detection microwell and is not released as waste through the size exclusion filter. In non-limiting embodiments or examples, the affinity agents for capture of target analyte is included in the sensor with the electrochemical reporter of the product identity and integrity.

In non-limiting embodiments or examples, detection of an electrochemical reporter of the product and sample identity and integrity occurs after exposure to a sample or liquid which results in a signal of the electrochemical reporter. The observed signal is compared to the expected signal as an indicator of the integrity and identity of the product and sample as being capable of producing results. If the product is not identified as the expected product for the analyzer, a result is not allowed to be generated. If the product is does not have integrity such as product which is damaged or degraded, a result is not allowed to be generated. If the sample is not identified as the expected sample type to be use for the analyzer, for example a urine when a blood sample is expected, a result is not allowed to be generated. If the sample is does not have integrity such as sample which is adulterated or degraded, a result is not allowed to be generated. In other non-limiting embodiments or examples, detection of an electrochemical reporter of the product identity occurs before or after exposure to a sample and is indicative of the integrity of the product and sample as being capable of producing results suitable for linking the results to additional data.

In non-limiting embodiments or examples, additional data is the identity of the sample and donor, or previous results or data collected on the sample and donor or additional results collected on the sample and donor in the future. In non-limiting embodiments or examples, additional data is factory calibration setting. In non-limiting embodiments or examples, additional data is connected using near field communication (NFC) or Radio-frequency identification (RFID), Bluetooth, or wireless communication which requires a confirmation of detection of an electrochemical reporter of the product and sample identity before additional data can be utilized. In non-limiting embodiments or example this factory setting resides on a memory storage device on the sensor array.

In non-limiting embodiments or examples, a lack of confirmation of product and sample identity by detection of an electrochemical reporter prevents communications of additional data to be utilized. In non-limiting embodiments or examples, the break in communications of additional data is caused by a change of voltage or current to electrochemical reporters which prevents their operation.

Further non-limiting embodiments or examples are set forth in the following numbered clauses:
Clause 1: A method of automatic verification of the product and sample integrity and identity comprising: introducing a sample into one or more microwells; generating an electrochemical response of an electrochemical reporter and analyte detection reagent; measuring the electrochemical response; and determining an identity of the sample based on the electrochemical response and a known response.
Clause 2: The method of clause 1, wherein the electrochemical response is measured in the one or more microwells.
Clause 3: The method clause 1 or 2, further comprising comparing the electrochemical response to a known electrochemical response.
Clause 4: The method of any of clauses 1-3, further comprising comparing the electrochemical response to a second known electrical response; and determining an integrity and identity of sample and product based on the second electrochemical response and a known electrochemical response.
Clause 5: The method of any of clauses 1-4, further comprising producing immunoassay results with electrochemical responses.
Clause 6: The method of any of clauses 1-5, wherein the one or more microwells comprise a size exclusion filter.
Clause 7: The method of any of clauses 1-6, further comprising applying a current and voltage to the one or more microwells to prevent generation of electrochemical response.
Clause 8: The method of any of clauses 1-7, wherein the immunoassay results comprise quantitative sample enumeration.
Clause 9: The method of any of clauses 1-8, further comprising introducing the electrochemical reporters into one or more microwells, wherein the electrochemical reporters bind to the one or more microwells.
Clause 10: The method of any of clauses 1-9, further comprising introducing signal generating reagents, wherein the electrochemical response is generated when the signal generating reagents are converted into electrochemical response.
Clause 11: The method of any of clauses 1-10, wherein electrochemical reporters change in response to exposure to the sample.
Clause 12: The method of any of clauses 1-11, wherein differing concentrations of electrochemical reporters are introduced into each of the one or more microwells.
Clause 13: The method of any of clauses 1-12, further comprising calibrating the one or more microwells based on the electrochemical response in the one or more microwells and the known electrochemical response.
Clause 14: The method of any of clauses 1-13, further using the determination of identity and integrity as a criteria for allow the addition of additional data to the data generated by our device.
Clause 15: The method of any of clauses 1-14, further comprising passing product and sample integrity and identity to allow additional data to be added.

These and other features and characteristics of the present disclosure, as well as the methods of operation and functions of the related elements of structures and the combination of parts will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the disclosure.

### BRIEF DESCRIPTION OF THE DRAWINGS

FIG. 1 shows a schematic view of the invention for analysis of a sample where a linkage arm (1) can capture an analyte (2) by an affinity agent (3) either directly attached to the linkage arm (1) or bound to the linkage arm (1) via a high affinity capture agent (4). The linkage arm (1) is further attached to the micro-filtration sensor (5) in a microwell (6) with an electrode (12). The analyte (2), such as a cell or biomolecule, is captured by an affinity agent (3) after the addition of the sample (7) and not released as waste (9) through the micro-filtration sensor (5). A second affinity agent (8) for a target analyte detection is attached to a reagent capable of converting the signal generating reagent (10) into an electrochemical response (11). The electrochemical response (11) is measured with electrode (12) in the microwell (6) and converted to a measurement of the analyte (2).

FIG. 2 shows the electrochemical response (11) after addition of a sample by increased changes in current for increasing numbers of P. aeruginosa (PA) cells measured after high affinity capture of polyclonal antibody with biotin onto a neutravidin functionalized gold electrode surface and polyclonal antibody with alkaline phosphatase (ALP) and electrochemical response current signal is plotted versus the voltage and the responses are plotted for sample with 0, 5×10\hat{ }3, 10\hat{ }4, 2×10\hat{ }4, 3×10\hat{ }4, 4×10\hat{ }4, and 5×10\hat{ }4 PA cells/mL produced by p-aminophenol (pAP) generation.

FIG. 3 shows a schematic view of the invention for producing and reporting a result indicating the integrity and identity of a product and sample where a linkage arm (1) can capture an electrochemical reporter (13) of integrity and identity by a high affinity label and capture agent (4) or through being directly attached to the linkage arm (1). The linkage arm (1) is further attached to the micro-filtration sensor (5) in a microwell (6) with an electrode (12). The analyte (2), such as a cell or biomolecule, is not captured by an affinity agent after the addition of a sample (7), and is released as waste (9) through the micro-filtration sensor (5). A signal generating reagent (10) is added to the microwell (6) and the electrochemical response (11) to the electrochemical reporter (13) measured with the electrode (12) in the microwell (6).

FIG. 4 shows the electrochemical response (11) after addition of the sample (7) with increasing concentration of electrochemical reporter (13) captured high affinity by biotin onto a neutravidin linked to the micro-filtration sensor (5) in a microwell (6) with electrode (12). The electrochemical response (11) measured by plotting as current versus the voltage and for sample with 0, 32, 64, and 96 pM of alkaline phosphatase (ALP) as the electrochemical reporter and shows the electrochemical response (11) as changes in the current produced due to generation of p-aminophenol (pAP) as a signal generating reagent (10).

FIGS. 5A-5C show a scanning electrode microscope (SEM) view of the multiple analyte detection microwells (6) as a micro-filtration sensor (14) with each microwell (6) having a slotted membrane (15) as the size exclusion membrane in the bottom of each microwell (6). The size exclusion membrane is of sufficient pore size to allow released analyte (2) to pass. Each microwell (6) is connected to an electrode (12) in the microwell (6) via separated current line (16) which connects to leads for changing working voltage of any individual microwell (6). The bottom slotted membrane (15) is coated in gold to allow application of reference current. Assay signals are read by respective current signal vs. voltage in accordance with a non-limiting embodiment of the invention for electrochemical detection or optically by imaging. Application of current into the microwell (6) across the bottom slotted membrane (15) is coated in gold. The electrode (12) in the microwell (6) is used to break the linkage arm (1) for any individual microwell (6).

FIG. 6 shows a schematic view of a system using a vacuum pump (17) connected to a vacuum pump motor driver (18), pressure sensor (19), and programmable controller board (20) as the hydrodynamic force for capturing cells and biomolecules from a sample on a porous surface (14) with analyte detection microwells (6) and micro-filtration sensor (5). The micro-filtration sensor (5) is sealed to a waste containment area (21) using a assay cartridge (22) for holding the porous surface (14). The system includes fluidics for reactions with liquid reagents (23) with dispensers (24) positioned over the micro-filtration sensor (5) and feed liquid reagents (23) by programmable dispensing pumps (25). The vacuum pump motor driver (18), pressure sensor (19), and the programmable dispensing pumps (25) are connected to the programmable controller board (20) used to monitor and regulate the vacuum pressure for filtration by maintaining a user-defined pressure in the waste collection chamber (21) and dispensing liquid reagents (23) by the programmable dispensing pumps (25). Voltage is applied to micro-filtration sensor (5) through electrodes and current is read using as electrochemical signals using a electrochemical reader (26) to read the sensors and to measure the impedance, voltage and/or current across the working and reference/counter microelectrodes in the microwell (6).

FIG. 7 shows a schematic view of an electrochemical reporter of product identity (13) captured via a high affinity capture agent by a biotin onto a neutravidin microparticle (27). The electrochemical reporter of integrity and identity (13) on the neutravidin microparticle (27) is then filtered (28) into one or more microwells (6) of an array of possible microwells with each microwell (6) having a porous surface (14) in the bottom of each microwell (6) of sufficient pore size to allow released analyte (2) to pass but not the electrochemical reporter (13) on the neutravidin microparticle (27). The analyte (2), such as a cell or biomolecule, is not captured by the neutravidin microparticle (27) after the addition of the sample (7) due to lack of affinity capture agent (4) and is released as waste (9) through the micro-filtration sensor (5). A signal generating reagent (10) is added to the microwell (6) and the electrochemical response to the electrochemical reporter is measured with the electrode (12) in the microwell (6).

FIG. 8 shows a schematic view of micro-filtration sensor (5) in one microwell (6) for producing and reporting both results for measurement of the analyte and results indicating the integrity and identity of a product and sample where linkage arms (1) can capture an electrochemical reporter (13) of integrity and identity by a high affinity label and capture agent (4) or high affinity capture agent (4) for capture of analyte. The linkage arm (1) is further attached to the micro-filtration sensor (5) in a microwell (6) with an electrode (12). The analyte (2), such as a cell or biomolecule, or electrochemical reporter (13) that is not captured by an affinity agent after the addition of a sample (7), and is released as waste (9) through the micro-filtration sensor (5). One or more signal generating reagent (10) are added to the microwell (6) and the electrochemical response (11) to the electrochemical reporter (13) and to the second affinity agent (8) for a target analyte detection and measured with the electrode (12) in the microwell (6).

FIG. 9 shows a schematic view of the invention for producing and reporting both results for measurement of the analyte and results indicating the integrity and identity of a product and sample where firmware on programmable controller board (20) operates an assay cartridge (22) by receiving user interface data (29) by touch screen (30) or by wireless communication (31) from a smart device application (32). The programmable controller board (20) activates the wireless read/writer (33) to read the read/write memory (34) on the assay cartridge (22) to provide assay cartridge data (35) to the programmable controller board (20). Potentiostat PCB (36) directly reads the analyzer calibration settings (37) from the PCB memory storage (38). The programmable controller board (20) calibrates the potentiostat PCB (36) with factory calibration setting from the assay cartridge data (35) and analyzer calibration setting (37) prior to measurement of the sample and then system is ready to produce the sensor readings (39) for assay cartridge (22). When the operational sequence is completed, the potentiostat PCB (36) captures the sensor readings (39) and generate the potentiostat output data (40) for a sample. The programmable controller board (20) uses the potentiostat output data (40) and any additional assay cartridge data (35) to calculate a result output records (41). Result output records (41) are sent to smart device application (32), touch screen (30) and to assay cartridge data (35).

### DESCRIPTION OF THE INVENTION

To promote an understanding of the principles of the invention, reference will now be made to the non-limiting embodiments illustrated in the drawings, wherein like reference numbers correspond to like or functionally equivalent elements, and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates. Certain embodiments of the invention are shown in detail, but some features that are well known, or that are not relevant to the present invention, may not be shown for the sake of conciseness and clarity. For purposes of the description hereinafter, the terms “end,” “upper,” “lower,” “right,” “left,” “vertical,” “horizontal,” “top,” “bottom,” “lateral,” “longitudinal,” “forward,” “reverse” and derivatives thereof shall relate to the example(s) as oriented in the drawing figures. However, it is to be understood that the example(s) may assume various alternative variations and step sequences, except where expressly specified to the contrary. It is also to be understood that the specific example(s) illustrated in the attached drawings, and described in the following specification, are simply exemplary examples or aspects of the invention. Hence, the specific examples or aspects disclosed herein are not to be construed as limiting. Moreover, as used in the specification and the claims, the singular form of terms include plural referents unless the context clearly dictates otherwise.

For purposes of the description hereinafter, the term integrity of product and sample means, for example, a product and sample capable of producing results and not having been compromised by conditions such as degradation, alteration, breakage, interference, or other factors that would alter results from expected values.

For purposes of the description hereinafter, the term identity of product and sample means, for example, source of these materials is determined as expected and is valid to be traceable to other data such as the collection location, manufacturing location, calibration setting, expected values, identity of sample donor, times of collection or manufacturing, and other data traceable to the materials.

An object of the disclosure, includes one or more electrochemical reporters of identity and integrity that are placed into a sensor array to produce an electrochemical response to the product and sample. The microfiltration sensor may be an array of analyte detection microwells which are also used for electrochemical detection of analytes with each of the one or more analyte detection microwells including a size exclusion filter, electrochemical detectors, and affinity agents for capture and detection of a target analyte. The electrochemical reporters do not interfere with the analysis of a target analyte, and are capable of generating an electrochemical reporter independent of a target analyte.

For purposes of the description hereinafter, an electrochemical reporter is a chemical that undergoes or catalyzes an oxidation and/or reduction during electrochemical reaction when placed between an anode electrode and a cathode electrode in an analyte detection microwell. Changes in current or resistance at defined voltages are used to detect the reduction or oxidation of the electrochemical response as a signal. Electrochemical reporters may include organic hydrocarbons and metals, which can be oxidized or reduced by accepting or donating electrons to change bond between atoms, such as C, O, N, S, P, H, and others, or valency of metals, such as +1, −1, etc. Electrochemical reporters may be chemicals such as enzymes, chelators, reductants, oxidants, mediators, and others can be used to enhance the signals produced.

For purposes of the description hereinafter, an analyte detection microwell for electrochemical detection of target analytes is as described in accordance with the IBRI PCT. The target analyte, size exclusion filter, detection microwell, electrochemical detector, and affinity agents for target analyte capture and detection are defined as terms and examples in accordance the IBRI PCT. The materials and methods described herein are useful with any of a broad variety of target analytes. The target analytes include a wide range of target molecules and target cells. In addition, the target analytes may comprise one or more target variants, as described hereafter.

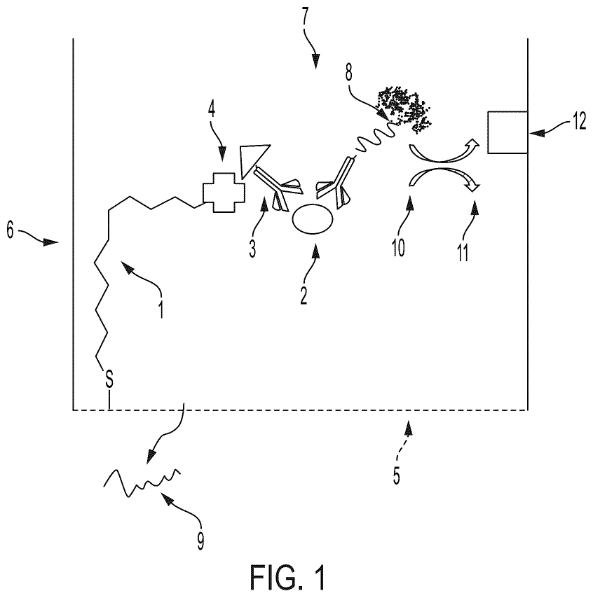

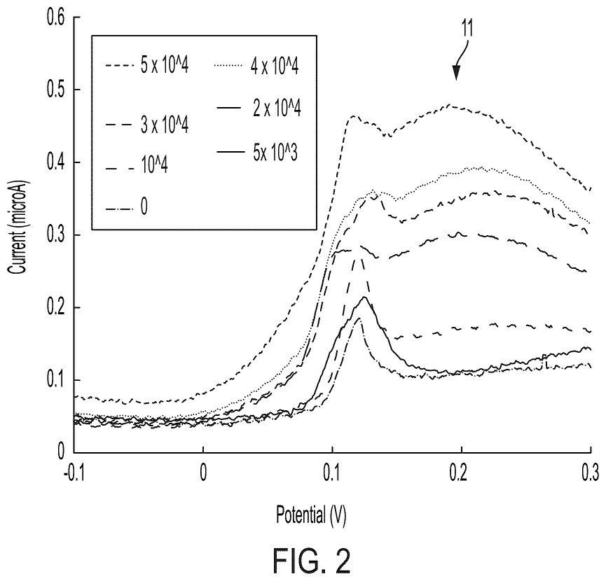

FIGS. 1 and 2 illustrate a non-limiting embodiment or example of the present disclosure, where an analyte (2) is detected in an analyte detection microwell (6) by electrochemical detection. The analyte detection microwell (6) includes a micro-filtration sensor (5), and affinity agents (3) for capture of the analyte (2) and a second affinity agent (8) for detection of the analyte (2). The affinity agent (3) for capture is attached to the microwell (6) by a linkage arm (1) directly or through a high affinity capture agent (4). A second affinity agent (8) for a target analyte detection is attached to a reagent capable of converting the signal generating reagent (10) into an electrochemical response (11). The affinity agent for target analyte for detection (8) produces an electrochemical response (11) after the addition of a sample (7) with the analyte (2) being measured, removing waste (9), and following the addition of a signal generating reagent (10). The electrochemical response (11) to the analyte (2) is measured with the electrode (12) in the microwell (6) and converted to a measurement of the analyte (2).

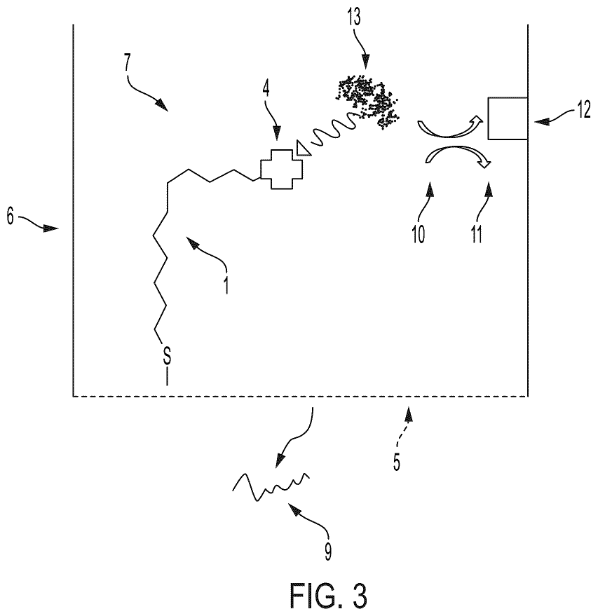

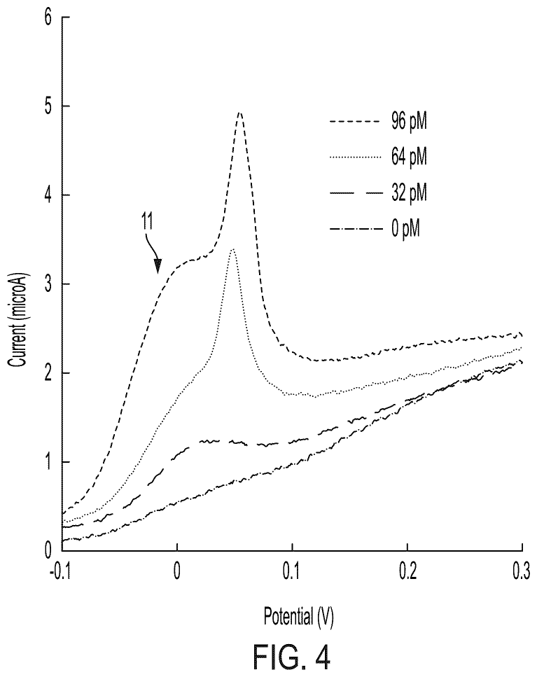

FIGS. 3 and 4 illustrate a non-limiting embodiment or example of the present disclosure, where an electrochemical reporter (13) is detected in a microwell (6) by electrochemical detection. The analyte detection microwell (6) includes a micro-filtration sensor (5) and electrode (12) for measuring the electrochemical reporter (13). The electrochemical reporter (13) is attached to the microwell (6) by a linkage arm (1) directly or through a high affinity capture (4). The electrochemical reporter (13) produces an electrochemical response (11) after addition of a sample (7) with the analyte (2) being removed as waste (8) and followed by the addition of signal generating reagent (10) to the microwell (6). The electrochemical response (11) is measured with an electrode (12) for the electrochemical detection and converted to a measurement of the electrochemical reporter (13) after exposure to the sample (7). The electrochemical reporter (13) measurements are compared with expected values as indication of the integrity and identity of the product and sample.

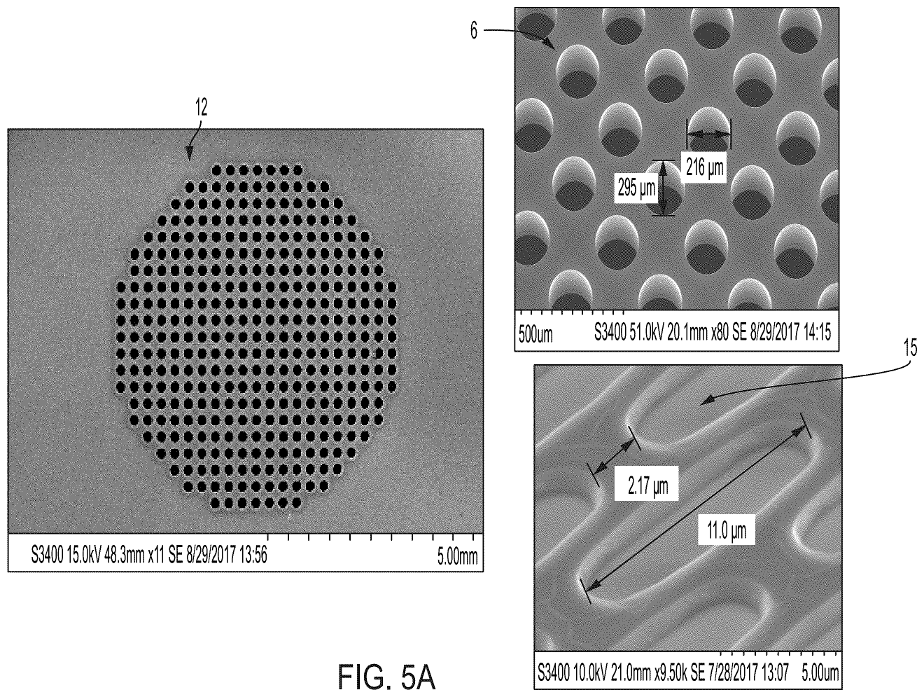

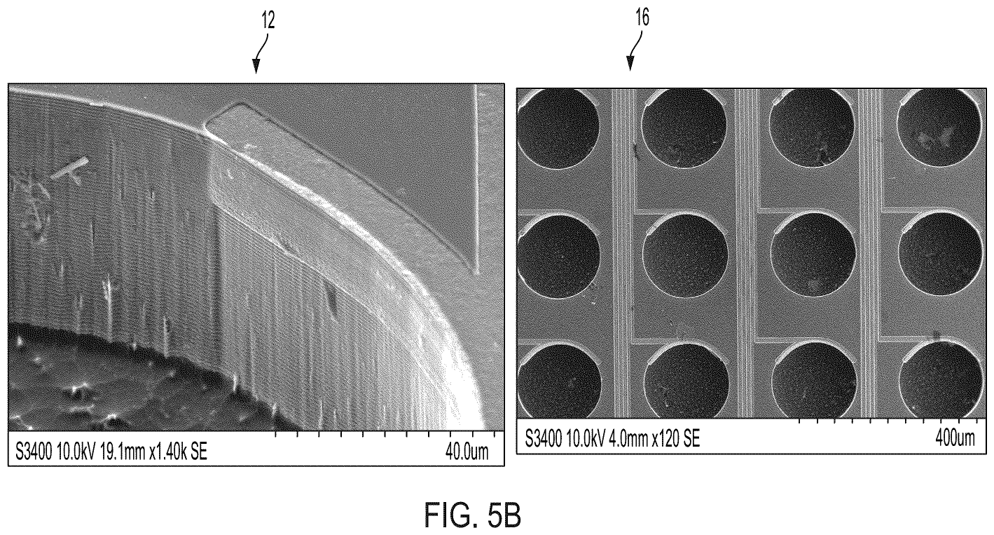

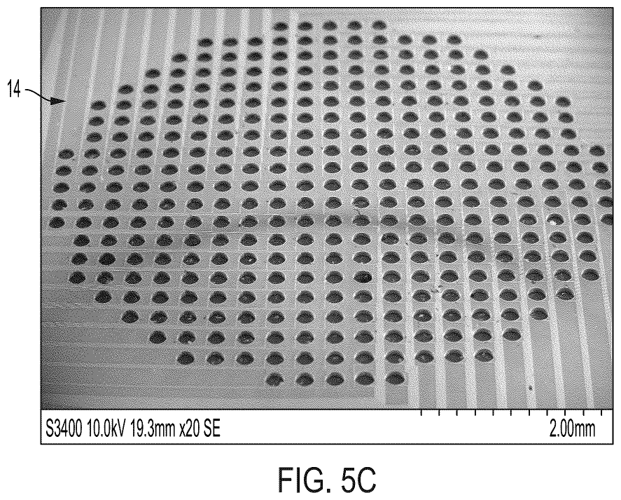

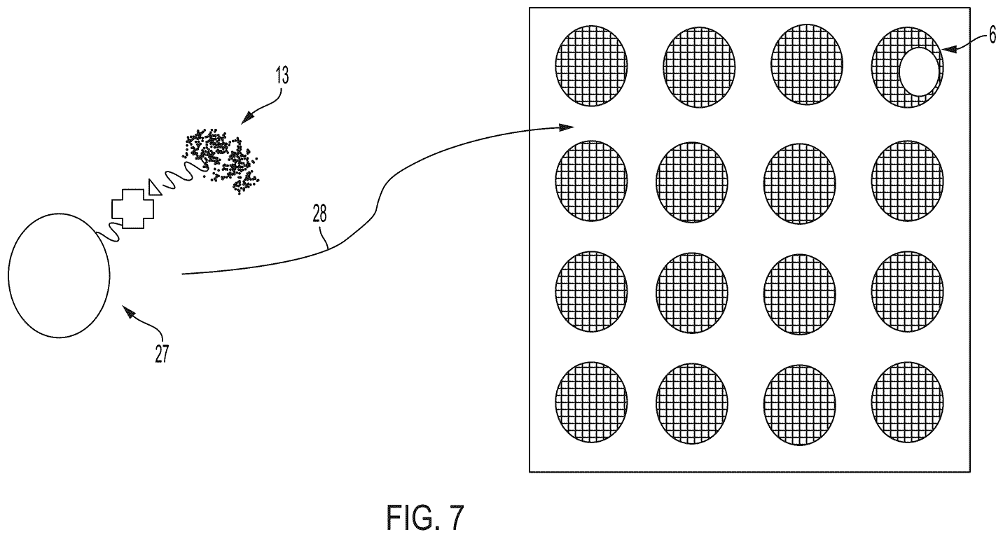

In non-limiting embodiments or examples, the measurements of the electrochemical reporter (13) is taken from one or more microwells (6) positioned in an array of multiple microwells (6) in a micro-filtration sensor (14) used for a set of assays for different markers, as shown for example in FIGS. 5 and 7. The system automatically identifies the expected location of the electrochemical reporter (13) for each microwell (6) in the array, and the measurements of the electrochemical reporter (13) are compared with expected measurements to indicate the integrity and identity of the product and sample.

In non-limiting embodiments or examples, once an acceptable identity and integrity of the product and sample have been confirmed by measuring the electrochemical reporter (13), analyte results are indicated as being suitable for linking additional information. Additional data is added to the measured set of analytes (2) by a microprocessor capable of linking data together such as in a programmable controller board (20). Examples of additional data can be any information, data, or record for the sample (7) tested such as the identity of the sample (7), sampling time, origin of the sample (7), such a patient, donor, organism, or environment, and other data connected to the sample (7). Additional data can be any information, data or record for the reagent and system used such as the identity of the system, location of the system, reagent lot, time and date of analysis, identity of the operator, calibration settings for the system and reagent and other data connected to the reagent and system.

In non-limiting embodiments or examples, the additional data linked is provided by a phone, smart device, clouds, hard disk, solid state disk, RFID encoders, NFC encoders, microprocessor chip (EEPROM), computers, voice, fingerprint, image, bar code, analyzer firmware, or other electronic methods. In some embodiments or examples, multiple data may be linked across a time period of the same origin. For example, the data can be linked to archived samples or to the same patient. The combined data can be connected over time to improve predictions of outcomes. For example, the data sets and analysis of biological material or biological system as signal, behavior, action, or characteristic of biological systems.

In practice, the invention can make use of the same signal generating reagent (10) used for the immunoassay electrochemical response (11) as the signal generating reagent (10) for the electrochemical reporter (13). For example, use of the electrochemical immunoassay (EC-IA), as previously described as an example (Pugia, Anal. Chem. 2021 or Pugia Anal Chem 2006), where enzyme, like alkaline phosphatase, is used to generate redox probe, like para-amino phenol as the electrochemical response (11) from an enzyme substrate, like para-amino-phenyl phosphate, as the signal generating reagent (10) or a nanoparticle is used a electrochemical response (11) from ferrous cyanide or other redox probes as the signal generating reagent (10). When enzymes, or nanoparticles are attached to a separate microwell (6), it can also be used as the electrochemical reporter (13) to generate electrochemical response (11) independent of the analyte (2). Both system, enzymes, and nanoparticles can be used in the same microwell (6), for electrochemical reporter (13) and analyte detection method (2) as long as the redox probe can be detected independently. These reagent methods can collect a sample and analyze the sample initially by reporting EC-IA results that are discussed in IBRI PCT.

### Example 1: Method to Automatically Identify Product and Sample Integrity for Data Linking

### Materials:

| | |
|---|---|
| Micro-filtration | 110 and 200 μm diameters and 300 μm depths were made using standard |
| sensors | microfabrication photolithography techniques. |
| Electrochemical | Biotinylated alkaline phosphatase (ALP-biotin) or alkaline phosphatase |
| reporter | (MW 85 kDa) or Biotinylated gold nanoparticle (NP-biotin) of 5 to 100 nm |
| | diameter. (Sigma Aldrich). |
| Bacterial cells | Polyclonal antibodies recognizing S. aureus (Thermo Fisher Scientific), E. |
| and antibodies | coli (MyBioSource, San Diego, CA, USA), K. pneumoniae (Thermo Fisher |
| | Scientific) and P. aeruginosa (Abcam, Cambridge, UK). |
| Attachment of | Antibodies separately conjugated to ALP (Thermo Fisher Scientific) using |
| reagents to | the FastLink ALP kit (Abnova, Taipei City, Taiwan) or NP (Thermo Fisher |
| affinity agents | Scientific), and to biotin-PEG4 and to Dylight 488 using the EZ-Link NHS- |
| | conjugation kits (Thermo Fisher Scientific). The resultant antibody |
| | conjugates were stored at 4° C. |
| Electrochemical | 1.05 mM solution of p-aminophenyl phosphate (pAPP, 3.0 mg, MW 189) |
| solution for ALP | in 100 mM TRIS, 600 mM NaCl, and 5 μM MgCl2 adjusted to pH 9.0. |
| Electrochemical | 100 mM KCl, 20 mM Fe(CN)₆³⁺ and 10 mM Fe(CN)₆⁴⁺ in TBS adjusted to |
| solution for | pH 8.0 |
| nanoparticle | |

Unless otherwise noted all other materials were purchased from Sigma Aldrich or Thermo Fisher Scientific.

### Example Method of Surface Capture in Microwells:

FIGS. 5A-5C illustrate a non-limiting embodiment of the present disclosure used for this example. This example method utilizes an array of micro-filtration sensors (5) each with porous surface (14) at the bottom (9.0×21.0 μm slot pores) with an overall pore space of 58 mm\hat{ }2 per well and size to pass analytes not selectively bound to a microwell (6). Additionally, each microwell (6) has an electrode (12) for routing the current into any given microwell (6). This array of microwells (6) was fabricated in silicon dioxide (SiO₂) by double polishing the silicon wafer base substrate (300 μm thick) to multiple micro-filtration sensors (6.5 mm diameter) of either 110 μm diameter or 200 μm diameter, and 300 μm depth.

Micro-filtration sensors (14) with arrays of microwells (6) of either 110 or 200 μm diameters and 300 μm depths were made using standard microfabrication photolithography techniques with <0.1 μm dimensional tolerance. Microwells (6) were patterned with the arrays inside a 6.5-mm diameter of 35 mm 2 or the size conventional ELISA plate well. In brief, film layers (4 to 20 μm) of dense, high-quality thermal SiO₂, Cu or Au were patterned with a slotted membrane (14) grid (9.0×21.0 μm pores) by photolithography and dry etch processes. A 200-nm layer of gold was added to the slotted membrane (15) grid by vapor deposition or gold coating to serve as a gold electrode. A second layer of 300 μm thickness was made with silicon (110 or 200 μm wells) by photolithography and dry etch processes order to create the array of microwells (6). The fabricated microwells layer was then mounted with the filtration membrane face up on the “top side” and was further processed for electrode with via gold electroplating of the micro-filtration sensor (5). The layers were mounted with the microwell (6) opening on the “top side” and was further processed by etching electrode current lines (16) and filling with copper via electroplating and covering the lines with a protective layer to keep each microwell (6) readable.

The neutravidin was linked to the gold surface of the slotted membrane (15) using the following functionalization procedure. The modification of the working electrode to functionalize the surface with neutravidin was performed by the 11-MUA, EDC, and HHSS method. This fabrication starts with dissolving 1.0 mM of 11-Mercapotundecanoic acid (11-MUA) into 50 mM phosphate buffer solution at pH 10. Next, 150 μL of the solution is added to each well and allowed to sit overnight. The wells were washed with water five times and heated at 37° C. until dry. The terminal carboxylic groups (of 11-MUA) were then activated for one hour by applying 150 μL of mM N-(3-dimethylaminopropyl)-N-ethylcarbodiimide hydrochloride (EDC) and 15 mM (N-hydroxy-succinimide ester (NHSS) in 50 mM phosphate buffer solution at pH 6.1. The sensor was washed with water five times and heated at 37° C. until dry. Next, the surface of the working electrode is treated with 0.5 μL of neutravidin (Thermo fisher Prod. 31000) dissolved at 10.0 mg/mL into 50 mM phosphate buffer and reacted for 30 minutes to immobilize at 37° C. until dry. The sensor was washed with water five times and heated at 37° C. until dry. In non-limiting examples, the neutravidin was replaced with alkaline phosphatase (1.7 mg/ml) and directly linked to the microwell.

After functionalization the micro-filtration sensors were blocked with 200 μL solution of blocking buffer. The blocking buffer was made with 112.5 mL of water containing 10% Candor (Candor Bioscience, Cat. #110125), 3.18 g MOPSO, 1.50 g BSA (Bovine Serum Albumin (Fraction V), and 60 uL Proclin 200 and adjusted to pH 7.5 with 10 N sodium hydroxide and the buffer. After blocking overnight, the micro-filtration sensors were washed five times with 200 μL of TBS-T (Tris buffered saline with 0.05% Tween-20) and allowed to air dry.

### Example Methods to Preform Analysis of Analyte

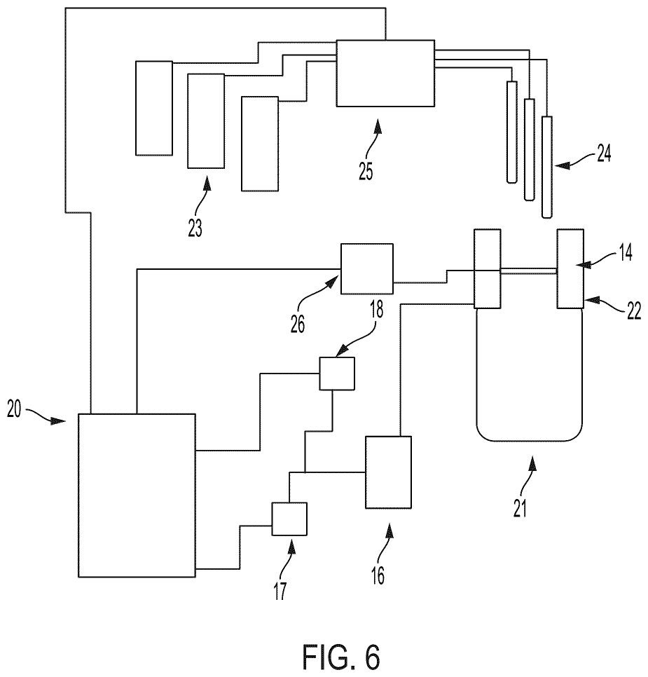

FIG. 6 illustrates a non-limiting embodiment of the present disclosure used in the example, whereby the micro-filtration sensor (14) with arrays of analyte detection microwells (6) is used as part of an analyzer. The analyzer uses vacuum filtration driven by an Arduino based proportional-integral-derivative (PID) controller logic to maintain the desired pressure in the waste collection chamber (21). It also drives the sample (7) and liquid reagent (23) fluids through micro-filtration sensor (5) placed in the bottom of each microwell (6) of the micro-filtration sensor (14) held in a assay cartridge (22). The analyzer uses a vacuum pump (17) as the hydrodynamic force for capturing analyte (2) or electrochemical reporter (13) on to the functionalized porous surface (14) in the microwells (6) of the micro-filtration sensor (5). Negative pressure for filtration was provided by vacuum pump (17) via underside of the micro-filtration sensor (5).

The analyzer included fluids for reactions with liquid reagents (23) and electrodes (12) and electrochemical reader (26) for detection of electrochemical responses (11) to the captured analyte (2) or electrochemical reporter (13). An Arduino controller with a menu-driven program (Adafruit Industries, New York, NY, USA) was used as the programmable controller board (20). A motor driver circuit board (18) was used to monitor and regulate the vacuum pressure for filtration (10-100 mbar negative pressure±10%). An MPXV5050DP analog differential pressure sensor (19) (Mouser Electronics, Mansfield, TX, USA) was used to measure the pressure in a conical 50-mL Falcon tube or 5-ml Eppendorf tube as the waste collection chamber (21). This Arduino-based vacuum-driven fluidic control system including proportional-integral-derivative (PID), maintained a user-defined pressure in the waste collection chamber (21). The control loop drives a DC diaphragm pump (22000.011, Boxer Pumps, Ottobeuren, Germany) through a DRV8838 brushed DC motor driver (Texas Instruments, Dallas, TX, USA) as the vacuum pump (17) to evacuate the air from the waste collection chamber (21). The vacuum pump (17) and the pressure sensor (19) were connected to waste collection chamber (21) using appropriate fluidic connectors (IDEX Health & Science, Oak Harbor, WA, USA). The dispensing of liquid reagents (23) was controlled using the same Arduino controller board (20) and three peristaltic dispensing pumps (24) with linear actuator motors to pump liquid reagents (23) into the micro-filtration sensor (14) for delivery (100 uL±1%) through steel needles as liquid dispensers (24).

For analysis of the analyte (2), biotinylated antibody reagents for capture and alkaline phosphate (ALP) labeled antibody reagents for detection in buffer are manually added to a complex sample containing the analyte (2) in a microwell (6). The antibodies used are specific for the analyte (2) to be detected. For example, polyclonal antibodies recognizing S. aureus (Thermo Fisher Scientific), E. coli (MyBioSource, San Diego, CA, USA), K. pneumoniae (Thermo Fisher Scientific) and P. aeruginosa (Abcam, Cambridge, UK) may be added. The analyte (2) for each example were bacterial lysate at 5×10\hat{ }3 to 5×10\hat{ }4 cell equivalents/mL prepared as described in Pugia Anal Chem 2021 and detected by square wave voltammetry as described on the micro-filtration sensor (5). In other examples the alkaline phosphate (ALP) labeled antibody reagents were replaced with nanoparticle (NP) labeled antibody reagents as described in Pugia Anal Chem 2006 and detected by electrochemical impedance spectroscopy (EIS) as described in Pugia (See Papers 1-3 Anal Chem 2006 etc) on the micro-filtration sensor (5).

### Example Methods to Preform Analysis of Electrochemical Reporters

As an example of analysis of electrochemical reporter (13), biotinylated alkaline phosphate (ALP) in buffer at 32, 64, and 96 pM are manually added to a complex sample (7) containing or lacking analyte (2) in a microwell (6). In this example, the electrochemical reporters (13) of different concentrations are kept separated in different microwells (6) and also are kept separated from the microwells (6) used for analysis of analytes (2).

The analyzer shown in FIG. 6 was used to automate: 1) drawing the complex sample with affinity agents (4,8) into a microwells (6) by turning the vacuum pump (17) on and off; 2) after incubation of antigen affinity complex in microwells (6) for <5 minutes the antigen affinity complex was captured by the neutravidin attached to micro-filtration sensor (5) through a linkage arm (1); 3) this was followed by addition and removal of 200 μL of TBS-T (Tris buffered saline with 0.05% Tween-20) as a wash solution with using the liquid dispensers (24) and pumps (25) to dispense TBS-T and the vacuum to remove TBS-T from the microwells (6) through the micro-filtration sensor (5) and followed by; 4) addition of a electrochemical solution containing para-amino-phenyl phosphate (pAPP) as the signal generating reagent (10) into the microwells (6). The presence of ALP generates para-amino phenol (pAP) as an electrochemical response (11) and allows potentiostat measurements with the electrochemical reader (26) by the electrodes (12) in each microwell (6). The electrochemical response (11) is detected by the electrodes (12) as a change in current as a function of voltage (FIGS. 2 and 4).

As another example of electrochemical reporter (13), biotinylated nanoparticles (NP) in buffer at 1, 3, and 10 pM are manually added to a complex sample (7) containing or lacking analyte (2) in a microwell (6). The electrochemical reporters (13) of different concentrations in this example are kept separated in different microwells (6) and also are kept separated from the microwells (6) used for analysis of analytes (2). The same analyzer process describe above is used, except that para-amino phenol (pAP) solution was changed to 0.5 mL Tween-20 10%, 0.625 mL of potassium ferricyanide 200 mM, 0.625 mL of potassium ferrocyanide 200 mM, and 2.5 mL of Tris-HCl 1 M pH 8 with ca. 15 mL of water in a 25 mL volumetric flask electrochemical response (11) and allows electrochemical impedance measurements with the electrochemical reader (26) by the electrodes (12) in each microwell (6).

### Example Methods to Prepare and Seed Electrochemical Reporters on Microparticle

Microparticle suspensions of 250 μL of 0.1% v/w streptavidin polystyrene beads (101 μm diameter; range of 90.0 to 105 μm; 0.03 mmol biotin FITC per mg) were added to 250 μL of blocking buffer and incubated at 37° C. overnight with constant shaking (˜800 rpm). The suspension was centrifuged at 1,000 rcf for five minutes to remove the supernatant, washed twice with 500 μL of PBS, and finally re-suspended in 500 μL TBS. The number of microparticles per μL was determined by phase contrast imaging (BioTek, Lionheart System). Additionally, the streptavidin polystyrene 2 microparticle could be replaced with streptavidin hydrogel particles, and 21 microparticle could additionally be optically labeled with Atto 550-Biotin or FluoSpheres™.

Blocked microparticles were captured into the blocked microwells (6) using the analyzer shown in FIG. 6 and was used to automate to drawing individual microparticles into microwells (6). The microparticles allowed capture of the antigen affinity complex by the streptavidin and eliminated the need for attaching neutravidin to micro-filtration sensor (5). The amount of particles (1% w/v) used to fill the microwells (6) (seeded) was optimized using the imaging result of micro-filtration sensor (14) wells after filtration. The amount added varied from 2 to 100 μL depending on the size and number of particles in microwells (6) present, and could be optimized such that over and under filling did not occur for ˜90% of microwells (6). In the case of 341 microwells of ˜120 μm diameter, individual microwell 100 μm neutravidin microparticle (27) could be filled into each microwell (6). Treatment with electrochemical reporter (ALP-biotin) at five levels, the concentrations were captured on the seeded microparticles by incubating for five minutes and washing twice with 500 μL of TBS solution for 43, 108, 240, 3288 fM per microparticle and microwell (6).

### Example Verification of Product and Sample Identity

According to a non-limiting example, affinity reagents (3 and 8) used for analyte (2) detection were added to assigned microwells (6) locations of the micro-filtration sensor (14). The electrochemical reporter (13) were loaded into the remaining microwells (6), where the locations of electrochemical reporters (13) were known at time of manufacture. All microwells (6) of the micro-filtration sensor (14) are able to process the sample (7) and generate an electrochemical signal and the electrochemical response (11) of these locations allowed comparison to expected values to identify micro-filtration sensor (14) product.

Each electrochemical reporter (13) can have unique electrochemical label concentration. For example, the four electrochemical reporter (13) concentrations as shown in FIG. 4. The assignment of the positions and concentrations for the electrochemical reporters (13) allow a unique 2D bar coding of each sensor, e.g. 4 variation for 96 microwells allows billions of codes. The codes of expected position and value can be known at the time of manufacture and, when verified, allow the diagnostic system to electronically load the manufacturing information and calibration codes to calculate the results.

FIG. 4 shows the electrochemical signal generated as current in μA plotted against the voltage (V) for the electrochemical reporter (13) captured by a high affinity capture agent (4) via a biotin onto a neutravidin linked to the micro-filtration sensor (5) in a microwell (6) with an electrode (12). FIG. 4 shows the electrochemical response (11) measured by plotting a current versus the voltage for samples with 0, 32, 64, and 96 pM of alkaline phosphatase (ALP) as the electrochemical reporter (13) and for current produced by p-aminophenol (pAP) generation. The response of the electrochemical reporter (13) is verified by comparing measured current against expected current. Observed values that match expected values indicate the product and sample data are suitable for linking to additional data such as manufacturing information and calibration codes that can be further used to calculate the results. Additional data connections can be linked between the results produced such as medical records, previous test results, patient, sample origin, time/date stamp, identity of, and archival sample among other examples of linkable data.

### Example Verification of Sample and Product Integrity

In non-limiting embodiments or examples, to verify samples integrity with analyte specific capture reagents, the response electrochemical reporters (13) exposed to the sample as well, and then compared against the value known at time of manufacture for a typical sample. If the response of the electrochemical reporters (13) were within an allowable range of the expected electrochemical response (11), the analyte results are calculated. When the electrochemical reporters (13) are impacted by the sample or product stability, then analyte results can be compensated for using the results of electrochemical reporters (13) as a live calibration. When the electrochemical reporters (13) are so impacted by the sample or product stability, that analyte results cannot be compensated then the sample is not viable and analyte results are prevented.

The calibration curve equation and correlation parameters at the time of manufacture are electronically uploaded for calculating results based on a typical sample and based on lot information to calculate the expected results using the electrochemical signals generated by analyte specific affinity reagents (3, 8) and the linked factory data.

The electrochemical reporters (13) exposed to the sample were used to correct the current for sample integrity when a difference in the sample background and response is observed from the typical sample. Since the electrochemical reporters and analyte specific capture reagents use the same electrochemical signal generating reagent (10), the electrochemical response (11) can be used to re-calibrate all analyte specific capture reagents and serve multiple analyte correction factors. Additionally, any impacted factor in the electrochemical reporters during the sample measurement will be corrected, such as temperature, degradation of reagent, humidity, and others.

FIG. 2 shows the electrochemical signal generated as current in μA plotted against the voltage (V) for the immunoassay detection (EC-IA) directly on the binding surface for samples including either 0, 5, 10, 20, 30, 40, or 50 thousand lysate equivalent of bacterial cells per assay. The immunoassay detection (EC-IA) directly on the binding surface achieved a quantitative bacterial immunoassay enumeration of cell counts across a range of 5,000 to 40,000 bacteria per sample increasing concentration of the analyte (2). This response was measured in five different urines of differing quality.

Sample variation is shown in Table 1 across the five different urines. In the middle column, only one calibration is used based on the typical sample and without use of electrochemical reporters (13). The observed variation across these samples is shown in Table 1 and ranged from 9% to 72% and indicate a lack of sample integrity for most samples. The same five urines were tested in the presence of the electrochemical reporter (13) and the observed electrochemical reporter (13) values were used to correct the calibration based on the typical sample. A significant improvement is shown by the reduction in variation (CV %) using the correction by the electrochemical reporters (13). After correction, all samples were comparable to a typical sample and had passing integrity.

TABLE 1
Impact of electrochemical reporter correction of samples

| | % CV for n-5 samples | % CV for n-5 samples with |
|---|---|---|
| Analyte level | without electro- | electrochemical reporters |
| (bacteria/mL) | chemical reporters | used to correct samples |
| 0 | 72% | 21% |
| 5 | 22% | 7% |
| 10 | 31% | 9% |
| 20 | 12% | 5% |
| 30 | 9% | 2% |
| 40 | 10% | 3% |
| 50 | 14% | 6% |

### Example of Method to Preform Analysis of Analyte and Electrochemical Reporter

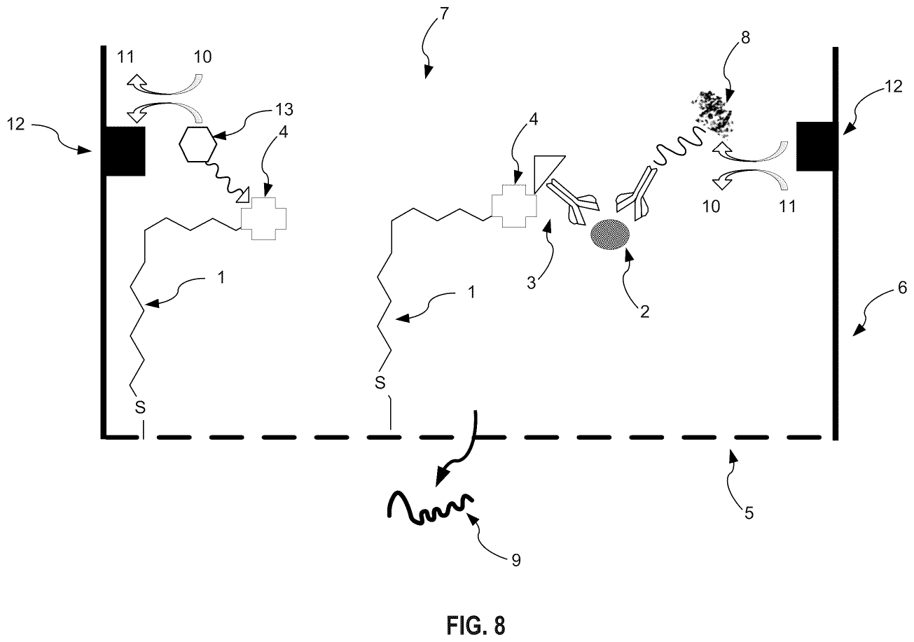

FIG. 8 illustrates a non-limiting embodiment of the present disclosure where by both analyte (2) and electrochemical reporter (13) can be detected on one micro-filtration sensor (5) in one microwell (6) for producing and reporting both results for measurement of the analyte and results indicating the integrity and identity of a product and sample where linkage arms (1) can capture an electrochemical reporter (13) of integrity and identity by a high affinity label and capture agent (4) or high affinity capture agent (4) for capture of analyte. The linkage arm (1) is further attached to the micro-filtration sensor (5) in a microwell (6) with an electrode (12). The analyte (2), such as a cell or biomolecule, or electrochemical reporter (13) that is not captured by an affinity agent after the addition of a sample (7), and is released as waste (9) through the micro-filtration sensor (5). One or more signal generating reagent (10) are added to the microwell (6) and the electrochemical response (11) to the electrochemical reporter (13) and to the second affinity agent (8) for a target analyte detection and measured with the electrode (12) in the microwell.

### Example Verification for Linking Data

When electrochemical reporters (13) demonstrate the integrity and identity of the product and sample is as expected then analytes results were produced and indicated as suitability for linking the results to other sample and product data. Additional data connections can be linked between the results produced such as medical records, factory calibration setting, previous test results, patient, sample origin, time/date stamp, identity of, and archival sample among other examples of linkable data. Additionally, when the electrochemical reporters (13) indicate poor integrity of sample or product or cannot identify the sample or product, suitability for linking the results to other sample and product data can be prevented. Voltage or current above the electrodes resistance will damage the electrode (12), electrochemical reporter (13) and signal generation of the second affinity agent (8). Current and voltage can be applied to the microwell to prevent to the microwell (6) from generating an electrochemical response (11) to the electrochemical reporter (13) and to the second affinity agent (8) for a target analyte detection. This failsafe operation that prevents downloaded of additional data.

### Example of Method to Operation of Data Linkage to Electrochemical Results

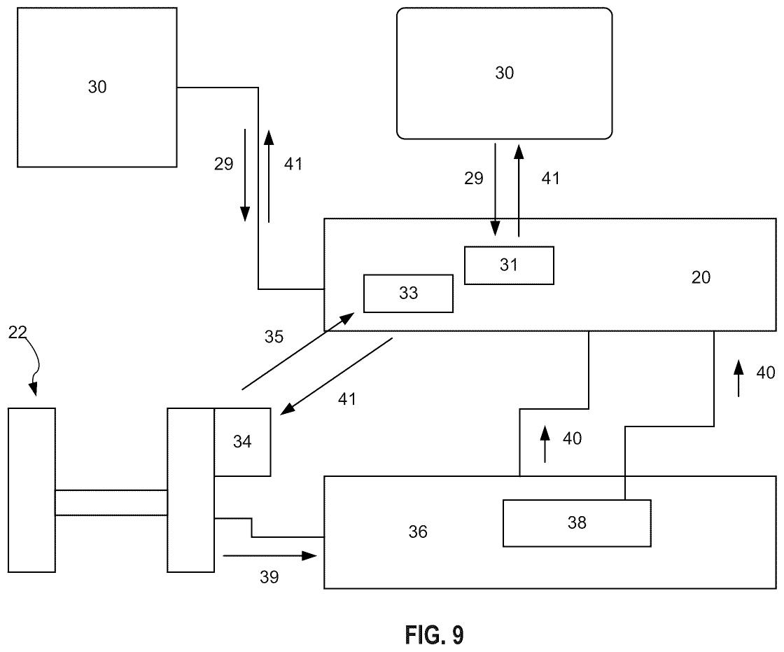

FIG. 9 illustrates a non-limiting embodiment of the present disclosure where by results for measurement of the analyte and results indicating the integrity and identity of a product and sample are linked to additional data. In this method the analyzer uses firmware on programmable controller board (20) to operates an assay cartridge (22) by receiving user interface data (29) by touch screen (30) or by wireless communication (31) from a smart device application (32). The programmable controller board (22) activates the wireless read/writer (33) to read the read/write memory (34) on the assay cartridge (22) to provide assay cartridge data (35) to the programmable controller board (20). This additional data can be medical records, factory calibration setting, previous test results, patient, sample origin, time/date stamp, identity of, and archival sample among other examples of linkable data. Potentiostat PCB (36) can also directly reads the analyzer calibration settings (37) from the PCB memory storage (38). Potentiostat PCB (36) is used to control electrodes and obtain the results by generating an electrochemical response (11) to the electrochemical reporter (13) and to the second affinity agent (8) for a target analyte detection The programmable controller board (20) calibrates the potentiostat PCB (36) with factory calibration setting from the assay cartridge data (35) and analyzer calibration setting (37) prior to measurement of the sample and then system is ready to produce the sensor readings (39) for assay cartridge (22). When the operational sequence is completed, the potentiostat PCB (36) captures the sensor readings (39) and generate the potentiostat output data (40) for a sample. The programmable controller board (20) uses the potentiostat output data (40) and any additional assay cartridge data (35) to calculate a result output records (41). Result output records (41) are sent to smart device application (32), touch screen (30) and to assay cartridge data (35).

In non-limiting embodiments or examples, to verify samples with analyte specific capture reagents, the response electrochemical reporters (13) exposed to the sample first, and then compared against the value known at time of manufacture for a typical sample. If the response of the electrochemical reporters (13) were within an allowable range of the expected electrochemical response (11), the analyte results are calculated.

The calibration curve equation and correlation parameters at the time of manufacture are electronically uploaded for calculating results based on a typical sample and based on lot information to calculate the expected results using the electrochemical signals generated by analyte specific affinity reagents (3, 8) and the linked factory data (FIG. 9). The electrochemical reporters (13) exposed to the sample were used to correct the current for sample integrity when a difference in the sample background and response is observed from the typical sample. Since the electrochemical reporters and analyte specific capture reagents use the same electrochemical signal generating reagent (10), the electrochemical response (11) can be used to re-calibrate all analyte specific capture reagents and serve multiple analyte correction factors. Additionally, any impacted factor in the electrochemical reporters during the sample measurement will be corrected, such as temperature, degradation of reagent, humidity, and others.

The electrochemical reporters (13) are demonstrated as indications of integrity and identity of the product and sample as the location and expected results were produced for the sample and reagent product when electrochemical reporters (13) were used. Expected results indicated the suitability for linking the results to other sample and product data. Additionally, and unexpectedly, the electrochemical reporters (13) allowed for improving samples of poor integrity to allow measurements of the analytes (2) by correcting calibration.

While the invention has been illustrated and described in detail in the drawings and foregoing description, the same is to be considered as illustrative and not restrictive in character, it being understood that only the representative embodiments have been shown and described and that all changes, equivalents, and modifications that come within the spirit of the inventions defined by the claims are desired to be protected. All publications, patents, and patent applications cited in this specification are herein incorporated by reference as if each individual publication, patent, or patent application were specifically and individually indicated to be incorporated by reference and set forth in its entirety herein.

1. A method of automatic verification of the product and sample integrity and identity comprising:
introducing a sample into one or more microwells;
generating an electrochemical response of an electrochemical reporter and analyte detection reagent;
measuring the electrochemical response; and
determining an identity of the sample based on the electrochemical response and a known electrochemical response.

2. The method of claim 1, wherein the electrochemical response is measured in the one or more microwells.

3. The method of claim 1, further comprising comparing the electrochemical response to a known electrochemical response.

4. The method of claim 1, further comprising
comparing the electrochemical response to a second known electrochemical response; and
determining an integrity and identity of the sample and product based on the electrochemical response and the second known electrochemical response.

5. The method of claim 1, further comprising producing immunoassay results with electrochemical responses.

6. The method of claim 1, wherein the one or more microwells comprise a size exclusion filter.

7. The method of claim 1, further comprising passing product and sample integrity and identity to allow additional data to be added.

8. The method of claim 1, further comprising applying a current and voltage to the one or more microwells to prevent generation of electrochemical response.

9. The method of claim 6, wherein the immunoassay results comprise quantitative sample enumeration.

10. The method of claim 1, further comprising introducing the electrochemical reporters into one or more microwells, wherein the electrochemical reporters bind to the one or more microwells.

11. The method of claim 1, further comprising introducing signal generating reagents, wherein the electrochemical response is generated when the signal generating reagents are converted into electrochemical response.

12. The method of claim 1, wherein electrochemical reporters change in response to exposure to the sample.

13. The method of claim 10, wherein differing concentrations of electrochemical reporters are introduced into each of the one or more microwells.

14. The method of claim 1, further comprising calibrating the one or more microwells based on the electrochemical response in the one or more microwells and the known electrochemical response.

15. The method of claim 1, further using the determination of identity and integrity as a criteria for allow the addition of additional data to the data generated by our device.
